(19) United States
(12) Patent Application Publication
(10) Pub. No.: US 2006/0105057 A1
(43) Pub. Date: May 18, 2006

(54) Compositions using tetrasilver tetroxide and methods for management of skin conditions using same

(75) Inventor: Marvin S. Antelman, Rehovot (IL)

Correspondence Address: BANNER & WITCOFF, LTD., 28 STATE STREET, 28th FLOOR, BOSTON, MA, 02109-9601, US

(73) Assignee: Marantech Holding, LLC, Warren, RI (US)

(21) Appl. No.: 11/318,648

(22) Filed: Dec. 27, 2005

Related U.S. Application Data
(63) Continuation of application No. 10/630,737, filed on Jul. 31, 2003, now abandoned.
Application No. 10/630,737 is a continuation of application No. 09/692,128, filed on Oct. 20, 2000, now abandoned.
Application No. 09/692,128 is a continuation-in-part of application No. 09/552,172, filed on Apr. 18, 2000, now Pat. No. 6,258,385.
(60) Provisional application No. 60/174,793, filed on Jan. 6, 2000.
(60) Provisional application No. 60/184,053, filed on Feb. 22, 2000.

(51) Int. Cl. A61K 33/38

(52) U.S. Cl. 424/618

(57) ABSTRACT

Pharmaceutical compositions including tetrasilver tetroxide (Ag₄O₄), such as in crystalline form, and methods of using such compositions for the prevention, treatment, and management various of dermatological skin conditions and diseases. In one embodiment, these compositions are substantially free of added persulfates. These dermatological conditions and diseases that may be prevented, treated, or managed with the compositions of the invention vary and include, but are not limited to, eczema, psoriasis, dermatitis, disease-induced skin ulcers, undefined tropical diseases, shingles, rashes, bedsores, cold sores, blisters, boils, herpes simplex, acne, pimples, skin chafing, skin cracking, itchiness, skin peeling, and warts.

### CROSS-REFERENCE TO RELATED APPLICATION

This application is a continuation of co-pending application Ser. No. 09/692,128, filed Oct. 20, 2000, which is a continuation-in-part of application Ser. No. 09/552,172, filed Apr. 18, 2000, and claims the benefit of Provisional Application Nos. 60/174,793, filed Jan. 6, 2000, 60/184,053, filed Feb. 22, 2000, and 60/214,503, filed Jun. 28, 2000.

### FIELD OF THE INVENTION

The invention relates to pharmaceutical compositions including tetrasilver tetroxide (Ag₄O₄) and methods of using such compositions for the prevention, treatment, and management of dermatological conditions or diseases.

### BACKGROUND OF THE INVENTION

Animal and mammalian skin, in particular, human skin, is a multifunctional organ. Not only does the skin provide an external covering to protect the body, but it also performs several specialized functions, such as breathing, perspiring, sensory information processing, and oil production. Oil production, essential to the protective features of the skin, works when an oily substance known as sebum is released from the sebaceous glands, which are large glands located at the base of a hair follicle. This permits the skin to moisturize and waterproof itself, thereby protecting itself from the environment.

The skin is the most environmentally-stressed organ in mammals, particularly in humans. The skin is subjected to toxic chemicals and hostile environments, as well as being the only organ directly exposed to Ultraviolet (“UV”) light in the presence of oxygen. Lengthy exposure of the skin to UV light typically damages the skin, resulting, in sunburn, photoaging, carcinogenesis, and other related skin disorders.

In particular, human skin is a composite material of the epidermis and the dermis. The topmost part of the epidermis is the stratum corneum. This layer is the stiffest layer of the skin, as well as the one most affected by the surrounding environment. Below the stratum corneum is the internal portion of the epidermis. Below the epidermis, the topmost layer of the dermis is the papillary dermis, which is made of relatively loose connective tissues that define the micro-relief of the skin. The reticular dermis, disposed beneath the papillary dermis, is tight, connective tissue that is spatially organized. The reticular dermis is also associated with coarse wrinkles. At the bottom of the dermis lies the subcutaneous layer.

The principal functions of the skin include protection, excretion, secretion, absorption, thermoregulation, pigmentogenesis, accumulation, sensory perception, and regulation of immunological processes. These functions are detrimentally affected by the structural changes in the skin due to aging and excessive sun exposure. The physiological changes associated with skin aging include impairment of the barrier function and decreased turnover of epidermal cells, for example.

The mechanical properties of the skin, such as elasticity, are believed to be controlled by the density and geometry of the network of collagen and elastic fiber tissue therein. Damaged collagen and elastin lose their contractile properties, resulting in skin wrinkling and skin surface roughness. As the skin ages or becomes unhealthy, it acquires sags, stretch marks, bumps, bruises or wrinkles, it roughens, and it has reduced ability to synthesize Vitamin D. Aged skin also becomes thinner and has a flattened dermoepidermal interface because of the alterations in collagen, elastin, and glycosaminoglycans.

UV light exposure in the presence of oxygen results in the undesirable creation of free radicals, which is believed to lead to various skin disorders, diseases, or conditions. In the skin, these free radicals frequently trigger the release of inflammatory mediators, commonly manifested as sun burn; cytoskeletal alterations, breaking down the collagen in the skin; and may also result in structural DNA changes, such as DNA strand breaks and dimer formation. The body attempts to neutralize the free radicals generated by UV light through the use of antioxidants. Antioxidants are commonly found in two forms—enzymatic and non-enzymatic. Conventional skin protection efforts typically attempt to either shield the skin from UV light to prevent the production of free radicals, or provide additional agents capable of neutralizing the free radicals.

Topical pharmaceutical applications are one such effort well known in the art that shields the skin from the sun's harmful effects. Sunscreens, for example, are used to protect the skin. Sunscreens are often water- or oil-based lotions or ointments that incorporate photo-protectant materials such as titanium and zinc oxide. Although the most widely used form of protection against exposure to sunlight, these topical applications suffer from several drawbacks. First, large amounts of photo-protective materials are incorporated into the topical applications, some of which have recently become suspect of having toxicity under these conditions or otherwise being harmful. Second, the effectiveness of such topical applications is dependent upon a constant and uniform coverage of the skin, which is often difficult to obtain. Many individuals fail to use these topical sunscreens on a regular or continuing basis, as is required to minimize damage to the skin under prolonged UV exposure. Third, sunscreens do not provide good protection for all types of UV light. Skin damage from UV exposure leads to a variety of dermatological disorders.

A variety of vitamins and minerals have individually been administered to treat certain skin and other problems that occur when the patient has a deficiency of that vitamin or mineral. Vitamin A, for example, assists in the treatment of acne and to facilitate wound healing; vitamin C (ascorbic acid) assists in the prevention of skin bruising and wound healing; vitamin E is an antioxidant; and copper assists in the treatment of elastic tissue defects. Topical use of vitamin C is also believed to ward off sun damage, reduce breakdown of connective tissues, and possibly promote collagen synthesis. Vitamin E is used topically as an anti-inflammatory agent, for enhancement of skin moisturization, for UV-ray protection of cells, and for retardation of premature skin aging. Catechin-based preparations, including proanthanols and proanthocyanidins are powerful antioxidants. These compounds are found in flowers, plant leaves, and grape seeds, for example.

Various of the above ingredients have been used alone or in certain combinations to form pharmaceuticals designed to prevent and treat certain cellular, skin, and other conditions. Although the above references disclose compositions and methods for treating various skin disorders, the treatments are often not completely effective and often involve adverse effects, such as overdrying of the skin. Furthermore, some existing treatments simply address the symptoms and fail to treat the underlying condition or disease, as well as helping to reduce the incidence of remission or the appearance of recurring or new disorders.

Multivalent silver molecules have also been disclosed for various uses, as they are reported to be non-toxic to animals and humans. M. Antelman, “Anti-Pathogenic Multivalent Silver Molecular Semiconductors,” Precious Metals, vol. 16:141-149 (1992); M. Antelman, “Multivalent Silver Bactericides,” Precious Metals, vol.16:151-163 (1992). For example, tetrasilver tetroxide activated with an oxidizing agent is disclosed for use in bactericidal, fungicidal, and algicidal use, such as in municipal and industrial water treatment applications and for the treatment of AIDS.

A variety of sources also report the use of certain divalent silver compounds for water treatment, as well as the use of such compounds, typically in combination with certain oxidizing agents, metals, or other compounds, as disinfectants, bactericides, algicides, and fungicides. One source also reports a single in vitro study of the use of such compounds for the treatment of AIDS. These sources include M. Antelman, “Silver (II, III) Disinfectants,” Soap/Cosmetics/Chemical Specialties, pp. 52-59 (Mar., 1994), and U.S. Pat. Nos. 5,017,295; 5,073,382; 5,078,902; 5,089,275; 5,098,582; 5,211,855; 5,223,149; 5,336,416; and 5,772,896.

U.S. Pat. No. 5,336,499 discloses tetrasilver tetroxide and persulfate compositions having certain in vitro anti-pathogenic properties, i.e., bactericidal, fungicidal, viricidal, and algicidal, in certain concentrations as low as 0.3 ppm, particularly in nutrient broth cultures. The persulfate or another oxidizing agent is required to activate the tetroxide crystals. Also disclosed are: an in vitro study regarding the inhibition of yeast growth in nutrient broth and the formulation of a gynecological cream and douche based on these results, and a report of an in vitro AIDS test with the compositions indicating total suppression of the virus at 18.0 ppm.

U.S. Pat. No. 5,571,520 discloses the use of molecular crystals of tetrasilver tetroxide, particularly with oxidizing agents to enhance the efficiency of such devices, for killing pathogenic microorganisms, such as staph infections. Amounts of 10 ppm sodium persulfate as an oxidizing agent were used with certain amounts of silver tetroxide in the reported in vitro testing. One human study involved in vivo curing of a gynecological yeast infection with 10 ppm of the silver tetroxide and 40 ppm sodium persulfate. Other in vivo topical studies report in conclusory fashion the cure of a single case of athlete's foot with a solution of 100 ppm of the composition and the cure of a single case of toenail fungus with a 25% suspension of the composition.

U.S. Pat. No. 5,676,977 discloses intravenously injected tetrasilver tetroxide crystals used for destroying the AIDS virus, AIDS synergistic pathogens, and immunity suppressing moieties (ISM) in humans. The crystals were formulated for a single injection at about 40 ppm of human blood. This reference also discloses the compositions cause hepatomegaly, also known as enlarged liver, albeit with no reported loss of liver function.

The aforementioned references report detailed descriptions of the mechanism via which the multivalent silver molecular crystal devices were believed to operate. The instant inventor also presented a discussion of such results and concepts at a Seminar entitled “Incurable Diseases Update” (Weizmann Institute of Science, Rehovot, Israel, Feb. 11, 1998). The title of this presentation was “Beyond Antibiotics, Non Toxic Disinfectants and Tetrasil™ (Trademark of applicant for the tetroxide).”

In this article, it was reported that the effects of the electron transfer involved with respect to the tetroxide, rendered it a more powerful germicide than other silver entities. The instant inventor holds patents for multivalent silver antimicrobials, e.g., U.S. Pat. Nos. 5,017,295 for Ag(II) and 5,223,149 for Ag (III); and while these entities are stronger antimicrobials than Ag (I) compounds, they pale by comparison to the tetroxide and so does colloidal silver that derives its germicidal properties from trace silver (I) ions it generates in various environments. Accordingly, the oligodynamic properties of these entities may be summarized as follows, which is referred to as the Horsfal series:
Ag₄O₄>Ag(III)>Ag(II)>>>>Ag(I)

The other unique property of the tetroxide was that it did not stain organic matter such as skin in like manner as Ag(I) compounds do. In addition, it was light stable.

Thus, it is desired to find pharmaceutical compositions and methods for preventing, treating, or managing one or more dermatological diseases or disorders. It is also desired to facilitate the prevention of future outbreaks of one or more disorders, as well as preventing, treating, and managing one or more dermatological disorders while avoiding the adverse effects present in many conventional dermatological treatments.

### SUMMARY OF THE INVENTION

The invention relates to pharmaceutical compositions including a therapeutically effective amount of tetrasilver tetroxide, or a pharmaceutically acceptable derivative thereof, substantially free of added persulfate. In one embodiment, the amount is from about 50 ppm to 500,000 ppm, while in another the amount is from about 400 ppm to 100,000 ppm. Optionally, the compositions include a carrier such that the composition is adapted for topical, parenteral, or transdermal administration. In a preferred embodiment, the carrier is adapted for topical administration. For example, the carrier can include petroleum jelly. In another embodiment, the compositions are adapted for topical administration and further include a thixotropic agent sufficient to increase adherence of the composition to skin to inhibit excessive runoff of the composition. This can facilitate administration of the proper dose to the patient. In another embodiment, the composition is prepared in the form of a powder or a plurality of powder crystals or granules.

The invention also relates to methods for preventing, treating, or managing one or more dermatological skin diseases in a patient's skin, which includes administering tetrasilver tetroxide, or a pharmaceutically acceptable derivative thereof, which is substantially free of added persulfate, to the skin in an amount and for a period of time which is therapeutically effective to treat such condition(s).

In one embodiment, the method further includes a carrier medium in which the tetrasilver tetroxide, or a derivative thereof, is dispersed, wherein the therapeutically effective amount is from about 50 ppm to 500,000 ppm, based on the weight of the carrier medium. In one embodiment, the carrier medium includes petroleum jelly. In another embodiment, the tetrasilver tetroxide, or a pharmaceutically acceptable derivative thereof, is administered in the form of a powder. In one embodiment, the therapeutically effective amount can be from about 400 ppm to 100,000 ppm. In varying embodiments, the composition may be administered in topical, parenteral, or transdermal form. In a preferred embodiment, the composition is topically administered directly to the skin. In yet another embodiment, the tetrasilver tetroxide composition, or a pharmaceutically acceptable derivative thereof, further includes a thixotropic agent sufficient to increase adherence of the composition to the skin so as to inhibit or prevent excessive runoff of the compositions from the skin.

In one embodiment, the skin disease being prevented, treated, or managed is caused by one or more autoimmune disorders rather than by a pathogen. In one embodiment, the skin disease is caused by a non-pathogenic condition comprising one or more of an autoimmune condition, a circulatory condition, or a neurological condition. In another embodiment, the skin disease prevented, treated, or managed includes at least one of eczema, psoriasis, dermatitis, ulcers, shingles, rashes, bedsores, cold sores, blisters, boils, herpes, acne, pimples, skin chafing, skin cracking, skin itch, skin peeling, heat rashes, leprosy, dermal tuberculosis, and warts. In a preferred embodiment, the disease prevented, treated, or managed includes one or more of cold sores, herpes, shingles, acne, psoriasis, dermatitis, skin ulcers, heat rashes, leprosy, dermal tuberculosis, or eczema. In a more preferred embodiment, the disease or condition is one or more of psoriasis, skin ulcers, heat rashes, leprosy, dermal tuberculosis, or atopic dermatitis.

In one preferred embodiment, the tetrasilver tetroxide, or a pharmaceutically acceptable derivative thereof, is completely free of added persulfate. In another embodiment, the administering includes application of the tetrasilver tetroxide, or a pharmaceutically acceptable derivative thereof, to the skin at a dosage level of about 10 mg to 500 mg per cm² of skin surface. In yet another embodiment, the therapeutically effective amount is insufficient to cause adverse effects.

The invention also relates to a method for preventing, treating, or managing one or more non-pathogenic, dermatological skin conditions, which includes administering tetrasilver tetroxide, or a pharmaceutically acceptable derivative thereof, to the skin in an amount and for a period of time which is therapeutically effective to treat such condition(s). In one embodiment, the non-pathogenic, dermatological skin condition includes an autoimmune disorder, a neurological condition, a circulatory condition, or a combination thereof.

### DESCRIPTION OF THE INVENTION

It has now been discovered that pharmaceutical compositions including tetrasilver tetroxide (Ag₄O₄) compounds as an active ingredient are advantageous in the prevention, treatment, and management of various indications. Preferably, the tetrasilver tetroxide compositions are substantially free of oxidizing agent, such as persulfate, since such compounds are believed to cause adverse effects, such as skin irritation and skin overdrying. More particularly, the invention relates to a method for treating dermatological conditions by applying a composition comprising tetrasilver tetroxide directly to the affected skin areas. In one embodiment, the compositions include a molecular scale device comprising at least one crystal of tetrasilver tetroxide. A plurality of these tetrasilver tetroxide molecules, such as on the order of trillions, may be employed in various pharmaceutical formulations and therapies to effectuate the prevention, treatment, and/or management of various dermatological conditions and diseases.

The dermatological conditions and diseases that may be prevented, treated, or managed with the compositions of the invention vary and include, but are not limited to, eczema, psoriasis, dermatitis, disease-induced or other skin ulcers, undefined tropical diseases, shingles, rashes, bedsores, cold sores, blisters, boils, herpes simplex, acne, pimples, skin chafing, skin cracking, itchiness, skin peeling, heat rashes, leprosy, dermal tuberculosis, and warts. In a preferred embodiment, the condition is one or more of psoriasis, skin ulcers, heat rashes, leprosy, dermal tuberculosis, or atopic dermatitis. Each condition should be understood as its own embodiment, although the present invention can certainly prevent, treat, or manage combinations of these conditions simultaneously.

In various embodiments, the dermatological conditions to be prevented, treated, or managed are non-bacterial, non-fungal, non-algal, or non-viral, or a combination thereof. The presently claimed invention is capable of treating dermatological conditions and diseases the cause of which is unknown at the present time. Nonetheless, the compositions and methods according to the invention may be employed to prevent, treat, or manage one or more of the above-noted diseases, and various conditions have indeed been treated clinically with notable effect. In one embodiment, the conditions are non-bacterial, non-fungal, non-algal, and non-viral, i.e., they have causes unknown to those of ordinary skill in the art at the present time and are not classified within these groups, such as by unknown pathogens of a different type, by autoimmune disorders, or by other means not within the four above-enumerated categories.

The compositions and methods of the invention advantageously prevent, treat, or manage dermatological diseases or conditions. “Management” includes controlling those dermatological conditions or diseases which cannot be cured completely, reducing the time of affliction of dermatological conditions or diseases, and the like. Preferably, the compositions prevent, treat, or manage dermatological conditions or diseases without visibly staining the skin, i.e., no staining to the naked eye. In one embodiment, the invention relates to the treatment or management, while in another embodiment the invention relates to the prevention, of dermatological diseases or conditions.

Without being bound by theory, it is believed that the crystal lattice of the Ag₄O₄ molecular device operates against pathogens by transferring electrons from its two monovalent silver ions to the two trivalent silver ions in the crystal, contributing to the death of pathogens by traversing their cell membrane surface. This in effect “electrocutes” the pathogens. The electrons are forced out of their balanced crystals by such labile groups as NH, NH₂, S—S and SH comprising pathogen cell membrane surface. Normal cells are not believed to be affected, because they are not believed to proliferate fast enough to expose these labile bonds. The K_{A} of Ag₄O₄ is 7.9×10⁻¹³, therefore the molecule is not believed to be disturbed unless more stable complexes are formed with such ligands as those comprising the pathogen cell membrane surface in a dynamic state. Indeed, the end result of the electron transfer, which is a redox reaction, is believed to result in the monovalent Ag ions being oxidized to Ag(II) and the trivalent Ag ions being reduced to the same end product, Ag(II). Accordingly, the well-known affinity of monovalent silver for certain elements such as sulfur and nitrogen is believed to be far exceeded here, for divalent silver is believed to not merely bind to these elements as does silver, but to actually form chelate complexes with their ligands. The molecular crystal attraction for the cell membrane surfaces is thus believed to be driven by powerful covalent bonding forces.

The electron transfer can be depicted by the following redox half reactions:
Ag⁺−e=Ag⁺²
Ag⁺³+e=Ag⁺²

It was found by rigorous testing that certain silver tetroxide containing-compositions were comparatively non-toxic compared to silver salts, such as conventional formulations of silver nitrate, silver sulfadiazine, and benzoyl peroxide. Since these silver tetroxide compositions were effective at certain ppm concentrations in killing pathogens in nutrient broth and for water treatment, commercial concentrates were formulated with 2% of the tetroxide. For acceptance of the oxide in commerce, for which EPA registration No. 3432-64 was obtained, it was necessary for the oxide to undergo a series of toxicity tests. A 3% concentrate was used and evaluated by a certified laboratory employing good laboratory practice (GLP) according to the Code of Federal Regulations for this purpose.

The results were as follows:

| | |
|---|---|
| Acute Oral Toxicity | LD₅₀ Greater than 5,000 mg/Kg |
| Acute Dermal Toxicity | LD₅₀ Greater than 2,000 mg/Kg |
| Primary Eye Irritation | Mildly irritating |
| Primary Skin Irritation | No irritation |
| Skin Sensitization | Non-Sensitizing |

Subsequent evaluations conducted according to the invention showed that unless persons were prone to silver allergies, the pure tetroxide compositions according to the invention could be applied to the skin without any ill effects or evidence of irritation, despite the fact that the compositions of the invention can be a powerful oxidizing agent. This can perhaps be explained by the stability manifested by the aforecited K_{A} of the compositions.

It was previously postulated, such as in earlier patents and/or literature relating to the various uses of certain silver tetroxide formulations, that it was required to use silver tetroxide in combination with an excess of a strong oxidizing agent, such as a persulfate, in order to effectively kill pathogens. It has now been found, however, that the additional presence of oxidizing agent(s) tends to be irritating to the skin. It has been found in accordance with the present invention that the additional oxide is not required and in some circumstances is undesirable for the purpose of treating the skin diseases described herein, in part due to the undesirable side effect of skin irritation when applied topically. Therefore, in one embodiment the present invention relates to compositions and methods of using the silver tetroxide compositions on the skin while minimizing the amount of additional oxidizer, such as persulfate. In one embodiment, the compositions are substantially free of added persulfates, while in a preferred embodiment, the compositions are completely free of added persulfates. In one preferred embodiment, the compositions are substantially free of added oxidizer, while in another preferred embodiment they are completely free of added oxidizer.

The tetrasilver tetroxide compound is black in color, such that care must be taken when formulating suitable topical pharmaceutical compositions according to the invention to inhibit or avoid blackening or superficially discoloring the skin. Without being bound by theory, it is believed that larger amounts of the silver tetroxide composition may induce increased superficial discoloration of the skin, or even skin staining. Thus, in one embodiment, the pharmaceutical compositions preferably have an insufficient amount of tetrasilver tetroxide composition to cause visible skin staining, more preferably an amount to reduce or avoid even superficial discoloration of the skin.

Where the tetroxide compositions according to the invention are applied to the skin, they may be combined with a carrier at an amount from about 5 ppm to 500,000 ppm, more preferably from about 50 ppm to 250,000 ppm of the tetroxide composition, based on the weight of the carrier. In various embodiments, the compositions are provided in amounts from about 400 ppm to 100,000 ppm, from about 1,000 ppm to 70,000 ppm, from about 10,000 ppm to 50,000 ppm, or from about 20,000 ppm to 40,000 ppm. In one preferred embodiment, the compositions are formulated with about 25,000 ppm to 35,000 ppm of tetrasilver tetroxide. It will be readily understood by those of ordinary skill in the art that 1 ppm of tetrasilver tetroxide composition is approximately equivalent to 1 mg/L for all metal oxides, such as tetrasilver tetroxide. The compositions, when applied topically, can be applied to the skin about 1 to 3 times per day until the condition is suitably cured or satisfactorily controlled. In one embodiment, the composition may generally be topically applied at a dosage level of from about 1 mg to 1000 mg per cm² of skin surface, preferably about 10 mg to 500 mg per cm² of skin surface.

The tetroxide compositions of the invention have been tested topically directly in powder form, as well as in several compounded formulations, for treating a wide assortment of skin conditions and diseases. Success was achieved in all cases except for certain stubborn nail fungi. A preferred carrier includes petroleum jelly, such as white petroleum jelly. For example, a suitable white petroleum jelly is available from Penreco of Houston, Tex.

The term “patient” as used herein refers to animals, particularly to mammals. In one preferred embodiment, the term patient refers to humans.

As used herein, the terms “adverse effects,” “adverse side effects,” and “side effects” include, but are not limited to, staining of the skin, superficial discoloration of the skin, headache, dry mouth, constipation, diarrhea, dry skin, hepatomegaly, fever, fatigue, and the like.

The phrase “therapeutically effective amount” when used herein in connection with the compositions and methods of the invention, means that amount of tetrasilver tetroxide composition, or a derivative thereof, which, alone or in combination with other drugs, provides a therapeutic benefit in the prevention, treatment, or management, of one or more of eczema, psoriasis, dermatitis, disease-induced skin ulcers, undefined tropical diseases, shingles, rashes, bedsores, cold sores, blisters, boils, herpes simplex, acne, pimples, skin chafing, skin cracking, itchiness, skin peeling, and warts, or one or more symptoms thereof. Different therapeutically effective amounts may be applicable for each disorder, as will be readily known or determined by those of ordinary skill in the art.

Tetrasilver tetroxide compounds for use according to the invention has been commercially sold under the poorly named “Ag(II) OXIDE” tradename. It may also be obtained from Aldrich Chemical Co., Inc., having a place of business in Milwaukee, Wis. The chemical synthesis of tetrasilver tetroxide compounds can be performed according to the method described on page 148 in M. Antelman, “Anti-Pathogenic Multivalent Silver Molecular Semiconductors,” Precious Metals, vol. 16:141-149 (1992) by reacting silver nitrate with potassium peroxydisulfate according to the following equation in alkali solutions:
4 AgNO₃+2 K₂S₂O₈+8 NaOH→Ag₄O₄+3 Na₂SO₄+K₂SO₄+2 NaNO₃+2 KNO₃+4 H₂O
To the extent necessary to understand the present invention, the disclosure of Antelman is hereby incorporated herein by express reference thereto.

The term “substantially free” means less than about 10 weight percent, preferably less than about 5 weight percent, more preferably less than about 1 weight percent, and most preferably less than about 0.1 weight percent of added persulfate is present according to the invention. In another embodiment, the term “substantially free” refers to the same amounts of added oxidizing agent present in the compositions.

The magnitude of a prophylactic or therapeutic dose of tetrasilver tetroxide composition(s), or a derivative thereof, in the acute or chronic management of diseases and disorders described herein will vary with the severity of the condition to be prevented, treated, or managed and the route of administration. For example, oral, mucosal (including rectal and vaginal), parenteral (including subcutaneous, intramuscular, bolus injection, and intravenous, such as by infusion), sublingual, transdermal, nasal, buccal, and like may be employed. Dosage forms include tablets, troches, lozenges, dispersions, suspensions, suppositories, solutions, capsules, soft elastic gelatin capsules, patches, and the like. The dose, and perhaps the dose frequency, will also vary according to the age, body weight, and response of the individual patient. Suitable dosing regimens can be readily selected by those of ordinary skill in the art with due consideration of such factors. In general, the total daily dosage for the conditions described herein, is from about 0.1 mg to 1,000 mg of the active ingredient, tetrasilver tetroxide, or a derivative thereof. In another embodiment, the daily dosage can be from about 1 mg to 500 mg, while in another embodiment, the daily dosage can be from about 2 mg to 200 mg of the tetrasilver tetroxide composition. A unit dosage can include, for example, 30 mg, 60 mg, 90 mg, 120 mg, or 300 mg of tetrasilver tetroxide composition. Preferably, the active ingredient is administered in single or divided doses from one to four times a day, such as by topical administration. In another embodiment, the compositions are administered by an oral route of administration. The oral dosage forms may be conveniently presented in unit dosage forms and prepared by any methods available to those of ordinary skill in the art of pharmacy.

In managing the patient, the therapy may be initiated at a lower dose, e.g., from about 0.05 mg, and increased up to the recommended daily dose or higher depending on the patient's global response. It is further recommended that children, patients over 65 years, and those with impaired renal or hepatic function, initially receive low doses when administered systemically, and that they be titrated based on individual response(s) and blood level(s). It may be necessary to use dosages outside these ranges in some cases, as will be apparent to those of ordinary skill in the art. Furthermore, it is noted that the clinician or treating physician will know how and when to interrupt, adjust, or terminate therapy in conjunction with individual patient response.

Any suitable route of administration may be employed for providing the patient with an effective dosage of tetrasilver tetroxide, or a derivative thereof. The most suitable route in any given case will depend on the nature and severity of the condition being prevented, treated, or managed.

In practical use, tetrasilver tetroxide, or a derivative thereof, can be combined as the active ingredient in intimate admixture with a pharmaceutical carrier according to conventional pharmaceutical compounding techniques. The carrier may take a wide variety of forms and may include a number of components depending on the form of preparation desired for administration. The compositions of the present invention may include, but are not limited to, suspensions, solutions and elixirs; aerosols; or carriers, including, but not limited to, starches, sugars, microcrystalline cellulose, diluents, granulating agents, lubricants, binders, disintegrating agents, and the like.

A preferred route of administration of the silver tetroxide compositions of the invention is topically, e.g., either directly as a powder or in non-sprayable or sprayable form. Non-sprayable forms can be semi-solid or solid forms including a carrier indigenous to topical application and preferably having a dynamic viscosity greater than that of water. Suitable formulations include, but are not limited to, suspensions, emulsions, creams, ointments, powders, liniments, salves and the like. If desired, these may be sterilized or mixed with one or more of any available auxiliary agents, carriers, or excipients, e.g., thixotropes, stabilizers, wetting agents, and the like, and combinations thereof. One or more thixotropic agents can be included in types and amounts sufficient to increase adhesion of topically applied compositions of the invention to the skin, so as to inhibit or prevent runoff or other loss of the composition from the treatment zone on the skin. Preferred vehicles for non-sprayable topical preparations include ointment bases, e.g., polyethylene glycol-1000 (PEG-1000); conventional ophthalmic vehicles; creams; and gels, as well as petroleum jelly and the like. In one more preferred embodiment, the carrier includes a petroleum jelly. In another preferred embodiment, the carrier is formulated as a cream, gel, or lotion. In another preferred embodiment, the carrier is 3 weight percent active ingredient, 36 weight percent heavy mineral oil, 47 weight percent petroleum jelly, and 14 weight percent Tivawax P, available from Tivian Laboratories, Inc., of Providence, R.I. In yet another preferred embodiment, the carrier may be a dry powder compositions, such as with 5 weight percent active ingredient and 95 weight percent bismuth subgallate. These topical preparations may also contain emollients, perfumes and/or pigments to enhance their acceptability for various usages.

Tetrasilver tetroxide, or a derivative thereof, may also be formulated for parenteral administration by injection (subcutaneous, bolus injection, intramuscular, or intravenous, such as by infusion), and may be dispensed in a unit dosage form, such as a multidose container or an ampule. Compositions of tetrasilver tetroxide, or a derivative thereof, for parenteral administration may be in the form of suspensions, solutions, emulsions, or the like, in aqueous or oily vehicles, and in addition to the active ingredient may contain one or more formulary agents, such as dispersing agents, suspending agents, stabilizing agents, preservatives, and the like.

In the case where an intravenous injection or infusion composition is employed, a suitable dosage range can be, e.g., from about 0.5 mg (0.1 ppm) to about 1,000 mg (200 ppm) total dose, preferably from about 5 mg (1 ppm) to 400 mg (80 ppm). In one preferred embodiment, the total dose can be from about 50 mg (10 ppm) to 200 mg (40 ppm). For intravenous injection, the concentrations stated should be understood to correspond to ppm of blood. It should be understood that any suitable amount of the composition according to the invention may be administered if effective to prevent, treat,. or manage one or more conditions described herein.

Pharmaceutical compositions of the present invention may be orally administered in discrete pharmaceutical unit dosage forms, such as capsules, cachets, soft elastic gelatin capsules, tablets, or aerosols sprays, each containing a predetermined amount of the active ingredient, as a powder or granules, or as a solution or a suspension in an aqueous liquid, a non-aqueous liquid, an oil-in-water emulsion, or a water-in-oil liquid emulsion. Such compositions may be prepared by any of the methods of pharmacy, but all methods include the step of bringing into association the active ingredient with the pharmaceutically acceptable carrier which constitutes one or more necessary ingredients In general, the compositions are prepared by uniformly and intimately admixing the active ingredient with liquid carriers or finely divided solid carriers or both, and then, if necessary, shaping the product into the desired presentation. Suitable types of oral administration include oral solid preparations, such as capsules or tablets, or oral liquid preparations. If desired, tablets may be coated by standard aqueous or nonaqueous techniques.

For example, a tablet may be prepared by compression or molding, optionally, with one or more accessory ingredients. Compressed tablets may be prepared by compressing in a suitable machine the active ingredient in a free-flowing form such as powder or granules, optionally mixed with a binder, lubricant, inert diluent, granulating agent, surface active agent, dispersing agent, or the like. Molded tablets may be made by molding, in a suitable machine, a mixture of the powdered compound moistened with an inert liquid diluent. In one embodiment, each tablet, capsule, cachet, or gel cap contains from about 0.5 mg to about 500 mg of the active ingredient, while in another embodiment, each tablet contains from about 1 mg to about 250 mg of the active ingredient. However, the amount of active ingredient found in the composition may vary depending on the amount of active ingredient to be administered to the patient.

Another suitable route of administration is transdermal delivery, for example, via an abdominal skin patch.

Tetrasilver tetroxide, or a derivative thereof, may be formulated as a pharmaceutical composition in a soft elastic gelatin capsule unit dosage form by using conventional methods well known in the art, such as in Ebert, Pharm. Tech, 1(5):44-50 (1977). Soft elastic gelatin capsules have a soft, globular gelatin shell somewhat thicker than that of hard gelatin capsules, wherein a gelatin is plasticized by the addition of plasticizing agent, e.g., glycerin, sorbitol, or a similar polyol. The hardness of the capsule shell may be changed by varying the type of gelatin used and the amounts of plasticizer and water. The soft gelatin shells may contain an additional preservative, such as methyl- and propylparabens and sorbic acid, to prevent the growth of fungi, although this is not necessary since the compounds and compositions of the invention provide anti-fungal efficacy. Thus, in one embodiment, the invention includes a compositions formulated as a gelatin shell with tetrasilver tetroxide, completely free of added preservatives. The active ingredient may be dissolved or suspended in a liquid vehicle or carrier, such as vegetable or mineral oils, glycols such as polyethylene glycol and propylene glycol, triglycerides, surfactants such as polysorbates, or a combination thereof.

In addition to the common dosage forms set out above, the compounds of the present invention may also be administered by controlled release means, delivery devices, or both, as are well known to those of ordinary skill in the art, such as those described in U.S. Pat. Nos.: 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,566, the disclosures of which are hereby incorporated herein by express reference thereto. These pharmaceutical compositions can be used to provide slow or controlled-release of the active ingredient therein using, for example, hydropropylmethyl cellulose in varying proportions to provide the desired release profile, other polymer matrices, gels, permeable membranes, osmotic systems, multilayer coatings, microparticles, liposomes, microspheres, or the like, or a combination thereof. Suitable controlled-release formulations available to those of ordinary skill in the art, including those described herein, may be readily selected for use with the tetrasilver tetroxide compositions of the invention. Thus, single unit dosage forms suitable for topical or oral administration, such as gels, lotions, cremes, tablets, capsules, gelcaps, caplets, and the like, that are adapted for controlled-release are encompassed by the present invention.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations may include: 1) extended activity of the drug; 2) reduced dosage frequency; and 3) increased patient compliance.

Most controlled-release formulations are designed to initially release an amount of drug that promptly produces the desired therapeutic effect, and gradual and continual release of other amounts of drug to maintain this level of therapeutic effect over an extended period of time. In order to maintain this constant level of drug in the body, the drug should be released from the dosage form at a rate that will replace the amount of drug being metabolized and excreted from the body.

The controlled-release of the active ingredient may be stimulated by various inducers, for example pH, temperature, enzymes, water, or other physiological conditions or compounds. The term “controlled-release component” in the context of the present invention is defined herein as a compound or compounds, including polymers, polymer matrices, gels, permeable membranes, liposomes, microspheres, or the like, or a combination thereof, that facilitates the controlled-release of the active ingredient (e.g., tetrasilver tetroxide) in the pharmaceutical composition.

The pharmaceutical compositions for use in the present invention include tetrasilver tetroxide, or a derivative thereof, as the active ingredient, and may also contain a pharmaceutically acceptable carrier, and optionally, other therapeutic ingredients. Suitable derivatives include any available “pharmaceutically acceptable salts,” which refer to a salt prepared from pharmaceutically acceptable non-toxic acids including inorganic acids, organic acids, solvates, hydrates, or clathrates thereof. Examples of such inorganic acids are nitric, sulfuric, lactic, glycolic, salicylic, and phosphoric. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic (besylate), stearic, sulfanilic, alginic, galacturonic, and the like. Particularly preferred acids are lactic, glycolic, and salicylic acids. The pharmaceutically acceptable salts preferably do not include halide-containing salts, as these are believed to facilitate breakdown of the oxide lattice present in the metal oxide compositions of the invention.

The term “about,” as used herein, should generally be understood to refer to both numbers in a range of numerals. Moreover, all numerical ranges herein should be understood to include each whole integer within the range.

### EXAMPLES

These and other aspects of the present invention may be more fully understood with reference to the following non-limiting examples, which are merely illustrative of the preferred embodiment of the present invention, and are not to be construed as limiting the invention, the scope of which is defined by the appended claims.

### EXAMPLE 1

### Method of Treating Dermatological Disease According to Present Invention

A female, age 28, resident of Central America, had a red rash caused by an unidentified dermatological tropical disease on her thigh. The condition was cured by a light dusting of 20 mg of Ag4O₄ compound in crystal form on the area. Similar occurrences in the past to the subject failed to be cured by other dermatological preparation sold as cures for said condition.

### EXAMPLE 2

### Method of Treating Fungal Infection According to Present Invention

A female, age 27, had a fungus infection in her navel. She was cured by direct application of 20 mg of Ag₄O₄ compound in crystal form to the affected area within 24 hours.

### EXAMPLE 3

### Method of Treating Herpes Simplex Sores According to Present Invention

A female in her early thirties had suffered from recurrent cold sores for five years. The subject stated in a written communication, “I have tried every over-the-counter medication for this ailment without even marginal success. I even tried the five times a day for five days herpes medication that my doctor prescribed with disappointing results.” Subject tried various concentrations of Ag₄O₄ dispersed in petroleum jelly. All formulations reduced the severity and duration of the herpes simplex. Subject was given a final formulation of 10,000 ppm Ag₄O₄ dispersed in white petroleum jelly, e.g., 1 weight percent of the tetrasilver tetroxide with 99 weight percent petroleum jelly. In many instances, quick application of the ointment upon the appearance of a cold sore resulted in disappearance of the cold sore the next day. Otherwise, if not caught quickly, sores were contained within 36 hours, which was a vast improvement over the previous treatments used by the patient.

### EXAMPLE 4

### Method of Treating Itch According to Present Invention

An 82-year-old female had suffered six months from an external vaginal itch which defied treatment. Application of Ag₄O₄ ointment dispersed in petroleum jelly (as described in Example 3) cured the condition.

### EXAMPLE 5

### Method of Treating Herpes Simplex According to Present Invention

Twenty-two samples of Ag₄O₄ ointment as in Example 3 were distributed to different individuals who were suffering from herpes simplex. Each applied the ointment. While there was no attempt made to record the exact condition and severity of the herpes subjects prior to treatment, all 22 cases were cured within 48 hours.

### EXAMPLE 6

### Method of Treating Shingles According to Present Invention

Having achieved success against herpes simplex, it was decided to test Ag₄O₄ ointment against shingles, which without being bound by theory is believed to be caused by herpes zoster. Accordingly, a 67-year-old male applied the ointment of Example 3 three times a day for two days, after which time the shingles condition was completely gone.

### EXAMPLE 7

### Method of Treating Acne According to Present Invention

Two individuals, one male, the other female, ages 33 and 48, who were suffering from external acne condition,.treated their skin three times a day with Ag4O₄ ointment prepared according to Example 3. The acne was completely cured after two days of applying the ointment.

### EXAMPLE 8

### Method of Treating Oral Viral Herpes According to Present Invention

Fifteen patients with an age ranging from 30 to 35 that were diagnosed as having oral viral herpes were arranged in two groups. Group I had five patients that suffered from severe oral viral outbreaks with a recurring frequency of 21-28 days. The sizes of the herpes sores ranged from 3.5 to 5 mm. Group II had ten patients who suffered from normal oral viral outbreaks with a recurring frequency of 28-42 days. The sizes of the herpes sores ranged from 1.25 to 1.75 mm. Both groups applied 50 to 200 mg of ointment containing 3 weight percent tetrasilver tetroxide with 97 weight percent petroleum jelly to the affected areas. Group I applied the ointment (within 12 hrs.) after the herpes sores broke through the skin and blistered. Group II was divided into two subgroups. Group IIA applied the ointment (within 12 hrs.) after the herpes sores broke through the skin and blistered. Group IIB applied the ointment 4-12 hrs. before the herpes sores broke through the skin and blistered. Application was twice daily. Patients reported daily on the pharmacological effects. Sizes of the herpes growth was observed on a daily basis for five days and frequency of reoccurrence was observed and recorded.

Summary of Results

Group I: Over a period of 24-48 hours, all of the patients observed the herpes sores regress and dry out. By day three, the sores were not visible and the skin was healed. All patients exhibited a longer recurrence time of 32-44 days, excluding one patient who did not have a recurrence for eight months. The sizes of the herpes sores upon recurrence were significantly smaller at 2.2 to 3.5 mm.

Group IIA: Over a period of 24-48 hours, all the patients observed the herpes sore regress and dry out. By the end of day three, the sores were not visible and the skin was healed. All patients exhibited a longer recurrence time from 34-55 days. The sizes of the herpes sores upon reoccurrence were significantly smaller at 0.8 to 1.4 mm.

Group IIB: Over a period of 12-24 hours all the patients observed that the herpes was retained and never broke through the skin as a blister. By the end of day two, there were no signs of herpes sores at all. There was not even the slightest amount of discomfort around the area where the blisters would have flourished. All patients exhibited a longer recurrence time from 36-62 days. The sizes of the herpes upon recurrence were 0.7 to 1.6 mm.

Conclusions

Tetrasilver tetroxide used as a topological ointment: (1) eliminated oral viral herpes sores within a period of 48 hours from the time of the first application; (2) extended the recurrence period of the viral herpes breakout cycle; and (3) prevented the herpes virus from breaking through the skin when used before an outbreak occurs.

### EXAMPLE 9

### Method of Treating Diabetes-Induced Foot Ulcers According to Invention

Twenty eight patients in the age group ranging from 45 to 65 having diabetes-induced foot ulcers were arranged in two groups. All of the patients were taking insulin injections and were diagnosed as Type I insulin dependent. Moreover, all of the patients had presented the diabetic foot condition for at least 10 days prior to treatment

Group I included fourteen patients where culture swabs of the ulcerated skin indicated the presence of bacteria (infection). Group II included fourteen patients where culture swabs of the ulcerated skin did not indicate the presence of abnormal amounts of bacteria (no infection).

The patients in each group were treated by applying 200 mg of a petroleum jelly containing 3 wt % tetrasilver tetroxide twice daily to the ulcerated sores for a 30-day period. Daily evaluations of the skin condition were conducted by a dermatologist.

Summary of Results

Group I: Within 48 hours of the onset of treatment, the sores on the feet of all patients began to dry out. After 72 hours, the ulcers on all patients started to heal at the borders. By the fourth day, inflammation of the diseased tissue eased, and by the sixth day the ulcers were completely dry with no surface secretions. By the tenth day, the ulcers on all patients feet had completely disappeared. Lab tests indicated no sign of infection on the feet of any patient by the tenth day.

Group II: Within 24 hours of the onset of treatment the sores on the feet of all patients began to dry out and heal at the borders with no secretion. By the third day, the sores on all patients were covered with new healthy tissue. By the tenth day, the ulcers had healed and completed the process of forming scar tissue by 80%. At day 14 of the treatment, all of the ulcers were 100% healed with no sign of infection.

Continuous monitoring of both groups over the 30-day period indicated no reappearance of the ulcers.

The above tests demonstrated that tetrasilver tetroxide treatment was effective in both curing infections associated with diabetes-induced ulcers and healing the ulcers themselves. Without being bound by theory, it is believed that the active tetroxide compositions of the present invention accelerated the neovascularization process of the affected tissue and facilitated the treatment.

### EXAMPLE 10

### Method of Treating Atopic Dermatitis According to Present Invention

Twenty patients ranging from age 8 months to 10 years were clinically diagnosed as suffering from atopic dermatitis involving inflamed lesions of the face and extremities, but without bacterial involvement. These patients were previously treated by the application of topical steroids to the affected skin areas, which was not effective and was discontinued before these trials began. The patients were divided into two groups.

Group I had ten randomly selected patients. A petroleum jelly containing 3 wt % tetrasilver tetroxide was applied at a dosage of about 100 mg to all affected skin areas of each patient twice daily for a period of five days. Daily evaluation of the skin condition was made by a dermatologist.

Group II was a control group of the remaining ten patients. This group was treated by twice daily application to the affected skin areas of about 100 mg of pure petroleum jelly, which was free of added tetrasilver tetroxide.

Summary of Results

Group I: Within 12 hours of the onset of treatment, the lesions on all patients began to show healing and drying and no longer exhibited prurito in the affected skin areas. Within 24 hours of the onset of treatment, signs of irritation of the skin areas had subsided. After 48 hours, signs of irritation had disappeared and the lesions were no longer visible. No side effects were reported. Treatment on all patients was discontinued after 5 days, but the group was assessed daily for any recurrence of the lesions. Two of the patients presented a reappearance of lesions by the twenty-fourth day, but these lesions were smaller and less irritating than the original lesions. Treatment was resumed on these two patients and after 24 hours the subsequent lesions had disappeared.

Group II: At 12 hours after the onset of the application of pure petroleum jelly to the affected skin areas, there were no signs of improvement of the skin. After 23-days, the injuries remained the same. After 29-days, the lesions gradually became more irritated with no sign of healing of the atopic dermatitis.

The above tests demonstrated that the tetrasilver tetroxide treatment was effective in most patients in healing atopic dermatitis within 24 hours of the commencement of treatment and appeared to halt the self-immunological reaction of atopic dermatitis at the local level, avoid the infections typically caused by this disease, and reduced the risk of new injuries during the treatment period. The present compositions were effective in reversing disease when it recurred, increasing the period of recession of the condition.

### EXAMPLE 11

### Method of Treating Psoriasis and Related Disorders According to Invention

Twenty four patients between the ages of 13 and 40 years were diagnosed as suffering from psoriasis, exhibiting irritation, scaliness and both the Auspitz sign and the Koebner phenomenon. All patients had been previously treated with topical steroids and were genetic transmitters of psoriasis. The patients were divided into two groups.

Group I had 12 patients where psoriasis was diagnosed less than 60 days prior to treatment. A petroleum jelly containing 3 wt% tetrasilver tetroxide of 200 mg was applied to affected skin areas twice a day over a 30-day period and each patient was evaluated by a dermatologist twice daily during the trial, with continued monitoring for the 30-day treatment period.

Group II had 12 patients who were diagnosed more than 60 days prior to treatment. Disease in this group was more severe than Group I and most had been suffering from psoriasis for many years, some exhibiting extensive disease on their backs. All had suffered from the disease since childhood. This group was treated by the same protocol as Group I, and was evaluated three times daily by a dermatologist.

Summary of the Results

Group I: By the tenth day of treatment, the psoriatic plates and inflamed areas of the treated skin started to heal. By the twentieth day, the Auspitz signs had disappeared on all patients. The papulo scale injuries were barely visible and the injured tissue had begun the process of granulation at the edges. By day 22, the inflammation changes within the plates were minimal. By day 27, the psoriatic plates present in the diseased skin of all patients had disappeared. By day 30, the psoriatic plates began the resolution process. By day 35, the skin on all patients appeared to be healed and the repigmentation process of the skin had been initiated.

Group II: By the twentieth day of treatment, the healing process on all patients had commenced as evidenced by the resolution of psoriatic plates and appearance of new tissue. All the plates were surrounded by new, healthy tissue, and a clear restitution process had begun. By day 28, the papulo scale injuries were of smaller sizes and the Auspitz signs were no longer visible. By day 30, the Koebner phenomenon had disappeared on all patients. By day 35, the psoriatic plates were very small and no longer visible on any patient.

The above test demonstrated that topical application of tetrasilver tetroxide to the affected skin areas of psoriasis sufferers effectively healed and/or controlled this disease, i.e., cured psoriatic plates and papulo scale injuries consistent with psoriasis diagnosis. The test also demonstrated that the recovery length is based on the extensiveness of the psoriatic injury. It is also believed that moisturizing cream or other lotion should accompany the application of the compositions of the invention when treating psoriasis, so as to help reduce or prevent dryness of the injured tissues.

### EXAMPLE 12

### Treatment of Tinea Versicolor According to the Invention

Twenty patients between the ages of 24 to 35 were clinically diagnosed as suffering from Pitiriasis Versicolor (Tinea Versicolor), based on microscopic tissue examination. The patients were divided into two groups.

Group I had ten randomly selected patients. A petroleum jelly containing 3 wt % tetrasilver tetroxide was applied at a dosage of about 100 mg to all affected skin areas of each patient twice daily.

Group II was a control group of the remaining ten patients. This group was treated by twice daily application to the affected skin areas of about 100 mg of pure petroleum jelly, which was free of added tetrasilver tetroxide.

Observations of both groups were made for seven days, with evaluations for a 30 day period to ensure there were no additional changes in the condition.

Summary of Results

Group I: Within 48 hours of the onset of treatment, the dark brown injuries on the patients started to discolor. By the fourth day, all dermic injuries from the neck, thorax, and stomach disappeared. By the fifth day, no skin injuries were visible, the skin being free and clear of any spots or marks caused by the disease. The patients were evaluated for the duration of the period, with no further changes reported.

Group II: Patients did not experience any changes in their condition over the 30 days. A microscopic test was made at the end of the 30 days, and the injuries were the same.

The above tests demonstrated that the tetrasilver tetroxide treatment was effective against Pityrosporun Orbiculare (Malassesia Furfur) fungus believed to be responsible for causing Tinea Versicolor.

Based on all of the test data described above, the healing mechanism associated with the use of tetrasilver tetroxide to treat and cure at least some skin diseases, without being bound by theory, appears to involve mechanisms other than merely inhibiting or killing pathogens and curing infections that tend to aggravate disease and retard the natural healing process. The data indicate that healing is brought about even in cases where no abnormal bacteria counts or infection is evident. This suggests that tetrasilver tetroxide may also act against auto-antibodies that trigger autoimmune reactions associated with diseased tissue, as well as against other non-pathogenic conditions or diseases, such as circulatory or neurological conditions or diseases.

Although preferred embodiments of the invention have been illustrated in the accompanying drawings and described in the foregoing Detailed Description, it will be understood that the invention is not limited to the embodiments disclosed, but is capable of numerous rearrangements and modifications of parts and elements without departing from the spirit of the invention. It will be further understood that the chemical and pharmaceutical details of the compositions and methods of prevention, treatment, or management herein may be slightly different or modified by one of ordinary skill in the art without departing from the claimed invention.

What is claimed is:

1. A pharmaceutical composition for preventing, treating or managing one or more dermatological skin conditions comprising a therapeutically effective amount of tetrasilver tetroxide substantially free of added persulfate, wherein the pharmaceutical composition further comprises a semi-solid or solid carrier medium that adheres to skin.

2. The pharmaceutical composition of claim 1, wherein the therapeutically effective amount is from about 50 ppm to 500,000 ppm.

3. The pharmaceutical composition of claim 1, wherein the therapeutically effective amount is from about 400 ppm to 100,000 ppm.

4. The pharmaceutical composition of claim 1, adapted for topical administration and wherein the carrier comprises petroleum jelly.

5. The pharmaceutical composition of claim 1, further comprising a thixotropic agent sufficient to increase adherence of the composition to skin without excessive runoff.

6. The pharmaceutical composition of claim 1, further comprising a powder or a plurality of powder crystals or granules.

7. A method for preventing, treating, or managing one or more dermatological skin diseases in a patient's skin, which comprises administering tetrasilver tetroxide which is substantially free of added persulfate, to the skin in an amount and for a period of time which is therapeutically effective to treat such condition(s), wherein the pharmaceutical composition further comprises a semi-solid or solid carrier medium that adheres to skin.

8. The method of claim 7, wherein the therapeutically effective amount is from about 50 ppm to 500,000 ppm, based on the weight of the carrier medium.

9. The method of claim 8, wherein the carrier medium comprises petroleum jelly.

10. The method of claim 7, wherein the tetrasilver tetroxide is a powder.

11. The method of claim 8, wherein the therapeutically effective amount is from about 400 ppm to 100,000 ppm.

12. The method of claim 8, wherein the administering is topical or transdermal.

13. The method of claim 12, wherein the composition is topically administered directly to the skin.

14. The method of claim 13, wherein the tetrasilver tetroxide composition further comprises a thixotropic agent sufficient to increase adherence of the composition to the skin without excessive runoff.

15. The method of claim 13, wherein the skin disease is caused by a non-pathogenic condition comprising one or more of an autoimmune condition, a circulatory condition, or a neurological condition.

16. The method of claim 7, wherein the skin disease prevented, treated, or managed comprises at least one of eczema, psoriasis, dermatitis, ulcers, shingles, rashes, bedsores, cold sores, blisters, boils, herpes, acne, pimples, skin chafing, skin cracking, skin itch, skin peeling, heat rashes, leprosy, dermal tuberculosis, and warts.

17. The method of claim 16, wherein the skin disease prevented, treated, or managed is one or more of cold sores, herpes, shingles, acne, psoriasis, dermatitis, skin ulcers, heat rashes, leprosy, dermal tuberculosis, or eczema.

18. The method of claim 17, wherein the disease is psoriasis, skin ulcers, heat rashes, leprosy, dermal tuberculosis, or atopic dermatitis.

19. The method of claim 7, wherein silver tetroxide is completely free of added persulfate.

20. The method of claim 7, wherein the administering comprises application of the tetrasilver tetroxide to the skin at a dosage level of about 10 mg to 500 mg per cm² of skin surface.

21. The method of claim 7, wherein an amount of persulfate is insufficient to cause adverse effects.

22. A method for preventing, treating, or managing one or more non-pathogenic, dermatological skin conditions, which comprises administering tetrasilver tetroxide substantially free of added persulfate, to the skin in an amount and for a period of time which is therapeutically effective to treat such condition(s).

23. The method of claim 22, wherein the non-pathogenic, dermatological skin condition comprises an autoimmune disorder, a neurological condition, a circulatory condition, or a combination thereof.

24. The method of claim 22, wherein the non-pathogenic, dermatological skin condition is selected from the group consisting of: eczema, psoriasis, dermatitis, heat rash, skin ulcer, bedsore, blister, boil, skin chafing, skin cracking, skin itch and skin peeling.

25. A pharmaceutical composition for preventing, treating or managing a non-pathogenic, dermatological skin condition comprising a therapeutically effective amount of tetrasilver tetroxide substantially free of added persulfate, wherein the non-pathogenic, dermatological skin condition is selected from the group consisting of: eczema, psoriasis, dermatitis, heat rash, skin ulcer, bedsore, blister, boil, skin chafing, skin cracking, skin itch and skin peeling.
